(19) United States
(12) Patent Application Publication
(10) Pub. No.: US 2026/0115016 A1
(43) Pub. Date: Apr. 30, 2026

(54) INFLATABLE MEDICAL DEVICE WITH AN ELECTRONIC PUMP DEVICE FOR REGULATING PRESSURE IN AN INFLATABLE MEMBER

(71) Applicant: Boston Scientific Scimed, Inc., Maple Grove, MN (US)

(72) Inventors: Brian P. Watschke, Minneapolis, MN (US); Natalie Ann Borgos, Roseville, MN (US); Sarah Prozeller, Boxboro, MA (US); Matthew Johnson, Lino Lakes, MN (US); James F. Hiebert, Delano, MN (US); Sean Thomas Horan, Minneapolis, MN (US); Jessica Williams, Marlborough, MA (US); Keith R. Maile, New Brighton, MN (US)

(21) Appl. No.: 19/367,286

(22) Filed: Oct. 23, 2025

Related U.S. Application Data
(60) Provisional application No. 63/713,377, filed on Oct. 29, 2024.

(51) Int. Cl. A61F 2/48; A61F 2/00

(52) U.S. Cl. CPC A61F 2/484; A61F 2/004; A61F 2/482; A61F 2250/0001; A61F 2250/0013

(57) ABSTRACT

An inflatable medical device may include an electronic pump device configured to transfer fluid between an inflatable member and a fluid reservoir. The electronic pump device is configured to execute operations, including receiving a target inflated pressure value, in response to an inflate signal to initiate an inflate state, inflating the inflatable member to the target inflated pressure value, determining a threshold level based on the target inflated pressure value, detecting, during the inflate state, whether a pressure of the inflatable member achieves the threshold level for at least a period of time, and in response to the pressure of the inflatable member achieving the threshold level for at least the period of time, deflating or inflating the inflatable member such that the pressure achieves the target inflated pressure value.

### CROSS REFERENCE TO RELATED APPLICATION

This application claims priority to U.S. Provisional Ser. No. 63/713,377, filed on Oct. 29, 2024, entitled “AN INFLATABLE MEDICAL DEVICE WITH AN ELECTRONIC PUMP DEVICE FOR REGULATING PRESSURE IN AN INFLATABLE MEMBER”, the disclosure of which is incorporated by reference herein in its entirety.

### TECHNICAL FIELD

This disclosure relates generally to an inflatable medical device with an electronic pump device for regulating pressure in an inflatable member.

### BACKGROUND

Some inflatable medical devices include a manual pump, physically operated by a user, to transfer fluid between an inflatable member and a fluid reservoir.

### SUMMARY

In some aspects, the techniques described herein relate to an inflatable medical device including: an electronic pump device configured to transfer fluid between an inflatable member and a fluid reservoir, the electronic pump device configured to execute operations, the operations including: receiving a target inflated pressure value; in response to an inflate signal to initiate an inflate state, inflating the inflatable member to the target inflated pressure value; determining a threshold level based on the target inflated pressure value; detecting, during the inflate state, whether a pressure of the inflatable member achieves the threshold level for at least a period of time; and in response to the pressure of the inflatable member achieving the threshold level for at least the period of time, deflating or inflating the inflatable member such that the pressure achieves the target inflated pressure value.

In some aspects, the techniques described herein relate to a method including: receiving a target inflated pressure value; in response to an inflate signal to initiate an inflate state, inflating an inflatable member to the target inflated pressure value; determining a threshold level based on the target inflated pressure value; detecting, during the inflate state, whether a pressure of the inflatable member achieves the threshold level for at least a period of time; and in response to the pressure of the inflatable member achieving the threshold level for at least the period of time, deflating or inflating the inflatable member such that the pressure achieves the target inflated pressure value.

In some aspects, the techniques described herein relate to an inflatable medical device including: an electronic pump device configured to transfer fluid between an inflatable member and a fluid reservoir, the electronic pump device configured to execute operations, the operations including: receiving a target deflated pressure value; in response to a deflate signal to initiate a deflate state, deflating the inflatable member to the target deflated pressure value; determining a threshold level based on the target deflated pressure value; detecting, during the deflate state, whether a pressure of the inflatable member achieves the threshold level for at least a period of time; and in response to the pressure of the inflatable member achieving the threshold level for at least the period of time, deflating or inflating the inflatable member such that the pressure achieves the target deflated pressure value.

### BRIEF DESCRIPTION OF THE DRAWINGS

FIG. 1A illustrates an example of an inflatable medical device for regulating pressure of an inflatable member according to an aspect.

FIG. 1B illustrates an example of the inflatable medical device with an external device and a provider platform according to an aspect.

FIG. 1C illustrates an example of the inflatable medical device with an external device associated with a clinician according to an aspect.

FIG. 1D illustrates an example of the inflatable medical device with an external device associated with an administrator according to an aspect.

FIG. 1E illustrates a diagram depicting inflation pressure and thresholds over time according to an aspect.

FIG. 1F illustrates a plurality of operating modes of an electronic pump device according to an aspect.

FIG. 2 illustrates an exploded view of an electronic pump device according to an aspect.

FIG. 3 illustrates a perspective of an inflatable penile prosthesis according to an aspect.

FIG. 4 illustrates an example of an artificial urinary sphincter device according to an aspect.

FIG. 5 illustrates a flowchart depicting example operations of regulating pressure in an inflatable medical device according to an aspect.

FIGS. 6A to 6E illustrate examples of a user interface of an application according to an aspect.

FIGS. 7A to 7E illustrate examples of a user interface of an application according to another aspect.

### DETAILED DESCRIPTION

This disclosure relates to an inflatable medical device having an electronic pump device for regulating pressure in an inflatable member. In some examples, the inflatable member includes an inflatable cuff configured to be implanted around the urethra. In some examples, the inflatable medical device is a penile prosthetic, and the inflatable member is a pair of cylinders. The inflatable medical device includes a fluid reservoir, an inflatable member, and an electronic pump device.

The electronic pump device may receive a target inflated pressure value and/or a target deflated pressure value. A target inflated pressure value may be a target pressure level of an inflate state. A target deflated pressure value may be a target pressure level of a deflate state. For example, when the inflate state is activated, the electronic pump device transfers fluid from the fluid reservoir to the inflatable member until the pressure of the inflatable member achieves the target inflated pressure value. When the deflate state is activated, the electronic pump device transfers fluid from the inflatable member to the fluid reservoir until the pressure of the inflatable member achieves the target deflated pressure value. The electronic pump device includes a pressure sensor configured to generate pressure readings about the pressure of the inflatable member, and the electronic pump device uses the pressure readings to activate or deactivate one or more pumps and one or more valves to transfer the fluid between the fluid reservoir and the inflatable member to achieve the target inflated pressure value or the target deflated pressure value.

The electronic pump device may receive the target inflated pressure value and the target deflated pressure value from a memory device of the electronic pump device. The target inflated pressure value and the target deflated pressure value may be settings that can be defined by a user (also referred to as a patient) and/or a clinician (also referred to as a physician). During the inflate state or the deflate state, the member's pressure may deviate from the target inflated pressure value or the target deflated pressure value by external forces, body movement, leakage from individual components of the electronic pump device, inadvertent over-inflation or under-inflation, and/or a user's pressure adjustments (e.g., manipulation) during the inflate state or the deflate state.

The electronic pump device includes a pressure controller configured to implement a pressure control system that can automatically activate the pump(s) to inflate or deflate such that the pressure of the inflatable member achieves the target inflated pressure value and/or the target deflated pressure value. In response to an inflate signal to initiate an inflate state, the pressure controller inflates the inflatable member to the target inflated pressure value. The pressure controller determines a first threshold level (e.g., an upper threshold) and a second threshold level (e.g., a lower threshold) for the inflate state based on the target inflated pressure value. For example, during the inflate state, the pressure controller may set the first threshold as a pressure value (e.g., +X %) that is greater than the target inflated pressure value, and the second threshold level as a pressure value (e.g., −X %) that is less than the target inflated pressure value.

In some examples, during the inflate state, a user may use a device control to adjust the pressure of the inflatable member. In response to the new pressure set point (e.g., due to user manipulation) being different from the target deflated pressure value by a threshold level, the pressure controller may update the target inflated pressure value as the new pressure set point, and then re-determine the first and second threshold levels based on the updated target inflated pressure value.

In response to the pressure of the inflatable member during the inflate state being equal to or greater than the first threshold for a period of time, the electronic pump device may activate one or more pumps to transfer fluid from the inflatable member to the fluid reservoir until the pressure achieves the target inflated pressure value or is between the first threshold and the second threshold. In response to the pressure of the inflatable member during the inflate state being equal to or less than the second threshold, the electronic pump device may activate one or more pumps to transfer fluid from the fluid reservoir to the inflatable member until the pressure achieves the target inflated pressure value or is between the second threshold and the first threshold. In response to the pressure of the inflatable member during the inflate state being detected as being between the first threshold level and the second threshold level, the pump(s) are deactivated, and the valve(s) are closed.

In response to a deflate signal to initiate a deflate state, the pressure controller deflates the inflatable member to the target deflated pressure value. The pressure controller determines a first threshold level (e.g., an upper threshold) and a second threshold level (e.g., a lower threshold) for the deflate state based on the target deflated pressure value. For example, during the deflate state, the pressure controller may set the first threshold as a pressure value (e.g., +X %) that is greater than the target deflated pressure value, and the second threshold level as a pressure value (e.g., −X %) that is less than the target deflated pressure value.

In some examples, during the deflate state, a user may use a device control to adjust the pressure of the inflatable member. In response to the new pressure set point (e.g., due to user manipulation) being different than the target deflated pressure value by a threshold level, the pressure controller updates the target deflated pressure value as the new pressure set point, and then re-determine the first and second threshold levels based on the updated target deflated pressure value.

In response to the pressure of the inflatable member during the deflate state being equal to or greater than the first threshold for a period of time, the electronic pump device may activate one or more pumps to transfer fluid from the inflatable member to the fluid reservoir until the pressure achieves the target deflated pressure value or is between the first threshold and the second threshold. In response to the pressure of the inflatable member during the deflate state being equal to or less than the second threshold, the electronic pump device may activate one or more pumps to transfer fluid from the fluid reservoir to the inflatable member until the pressure achieves the target deflated pressure value or is between the second threshold and the first threshold. In response to the pressure of the inflatable member during the deflate state being detected as being between the first threshold level and the second threshold level, the pump(s) are deactivated, and the valve(s) are closed.

The inflatable medical device includes a controller configured to wirelessly interact with the electronic pump device. In some examples, the controller includes a patient application executable by an external device. The patient application may detect a user command, and, in response to the user command being detected, the patient application may transmit the inflate signal to the electronic pump device. In some examples, the patient application may display a user interface with device controls that allow the user to interact with the electronic pump device. In some examples, in response to a selection of a first device control, the patient application may generate and send an inflate signal to the electronic pump device. In some examples, in response to a selection of a second device control, the patient application may generate and send a deflate signal to the electronic pump device. In some examples, the patient application may detect voice commands (e.g., audio signals) and may generate signals, including inflate and deflate signals based on the voice commands.

In some examples, the pressure controller is configured to detect a forced deflate event, and, in response to the forced deflate event, the pressure controller may initiate deflation of the inflatable member, e.g., to a target deflated pressure value. In some examples, the forced deflate event includes detection of a disconnection to a wireless connection between the controller and the electronic pump device.

In some examples, the electronic pump device operates in one of a plurality of operating modes, such as an active (e.g., normal mode), a storage mode, an end-of-life mode, and/or a disconnected mode. In some operating modes, certain features or capabilities may be limited. In some examples, the inflatable medical device when packaged is configured with the storage mode. An electronic pump device in the storage mode may disable wireless communications, disable operations of a pump and valve, etc. In some examples, to get the electronic pump device out of the storage mode, the electronic pump device is configured to detect the presence of a magnetic field. For example, a person may place a charger device or a magnet in proximity to the electronic pump device. In response to the detection of the presence of a magnetic field, the electronic pump device may switch the operating mode from the storage mode to another mode such as an active mode. In some examples, the magnetic field will induce the flow of current, e.g., to start charging the battery. In some examples, the detection of the flow of current (e.g., start of the charging of the battery) may switch the device operating mode from the storage mode to the active mode. In some examples, the electronic pump device may initiate a countdown timer, and, in response to the expiration of the countdown timer, may switch an operating mode to the end-of-life mode. In some examples, the countdown timer is initiated when the electronic pump device is taken out of the storage mode.

In some examples, the controller includes a clinician application executable by an external device, where the clinician application configured to display a user interface on a display of the external device, receive, via the user interface, a clinician-defined setting (also referred to as a user-defined setting where the user can be a clinician or a patient), and transmit the clinician-defined setting to the electronic pump device. For example, a clinician may use the clinician application to set certain settings for the electronic pump device. In some examples, the clinician application may receive patient and device data, which may be displayed on the user interface and/or stored on a provider platform (e.g., executing on a server computer) and/or stored on the electronic pump device.

In some examples, the clinician's external device is a managed device by a management engine of the provider platform. The clinician's external devices include a management agent configured to receive enforcement policies from the provider platform and apply the enforcement policies to manage computer functions of the external device. The management agent may control the installation of applications, implement security policies, manage notifications, access or deny certain web content.

In some examples, the inflatable medical device includes a provider application executable by a provider device. The provider application may provide an operation interface with the ability for an administrator to deploy software updates (e.g., new device firmware, update to the device firmware, security updates, etc.) on the electronic pump device, and/or to the patient and clinician applications. In some examples, the inflatable medical device includes a support interface with controls that enable an administrator to deactivate a patient application and/or a clinician application. In some examples, the support interface may be used to enter patient and equipment enrollment information on the provider platform and/or registering new clinician applications and/or clinician devices.

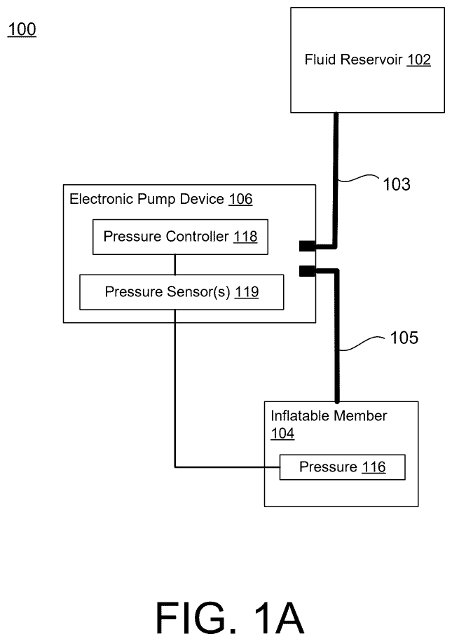

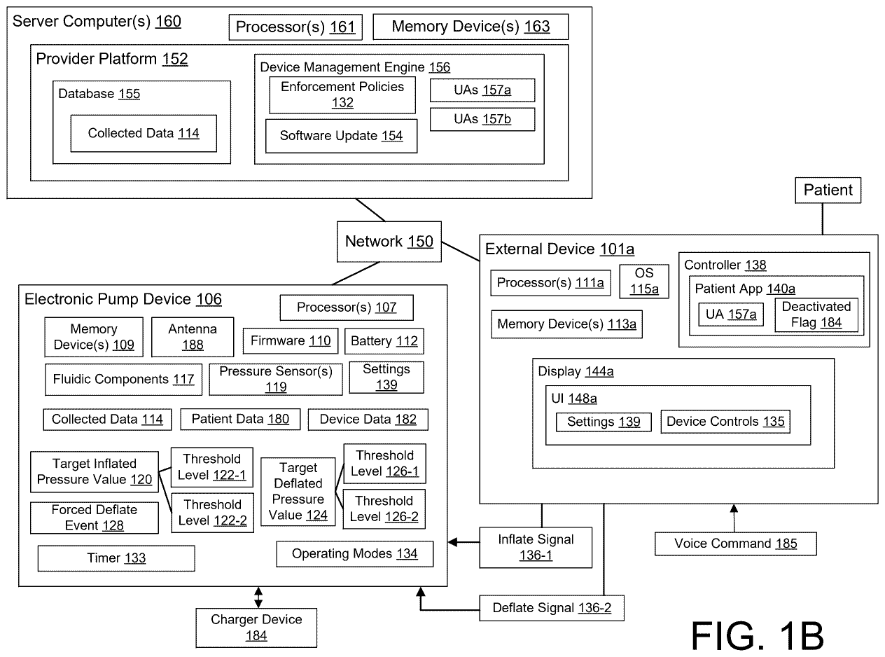

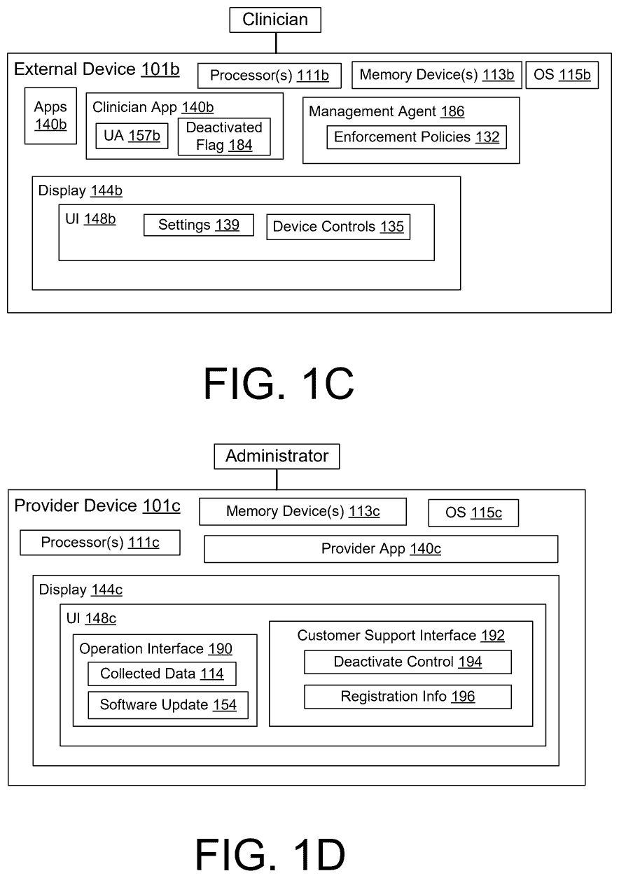

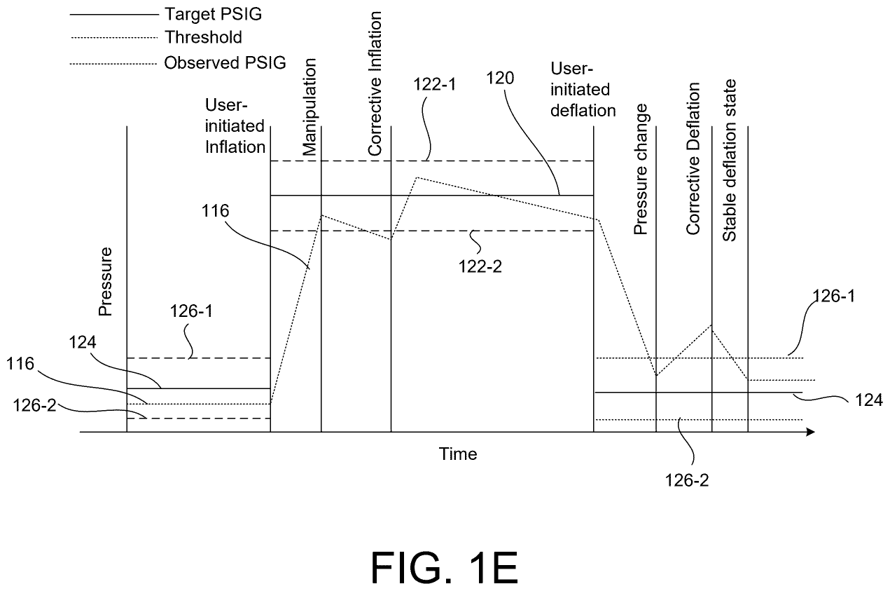

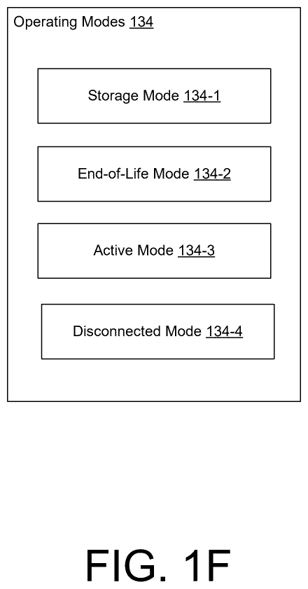

FIGS. 1A to 1F illustrates an inflatable medical device 100 according to an aspect. In some examples, the inflatable medical device 100 is an artificial urinary sphincter device. In some examples, the inflatable medical device 100 is an inflatable penile prosthesis. However, the inflatable medical device 100 may include any type of medical device that transfers fluid between components of the inflatable medical device 100. The inflatable medical device 100 includes a pressure controller 118 configured to manage a pressure 116 of the inflatable member 104 during an inflate state or a deflate state to automatically maintain a pressure 116 around a desired pressure level.

The inflatable medical device 100 includes a fluid reservoir 102, an inflatable member 104, and an electronic pump device 106 configured to transfer fluid between the fluid reservoir 102 and the inflatable member 104. In some examples, the inflatable member 104 is an inflatable cuff member configured to be implemented around a urethra of a patient. In some examples, the inflatable member 104 is a penile inflation member (e.g., one or more inflatable cylinders) that may be implanted into the corpus cavernosum of the user. The fluid reservoir 102 may be implanted in the abdomen or pelvic cavity of the user (e.g., the fluid reservoir 102 may be implanted in the lower portion of the user's abdominal cavity or the upper portion of the user's pelvic cavity). In some examples, at least a portion of the electronic pump device 106 may be implanted in the patient's body.

The inflatable member 104 may be capable of expanding upon the injection of fluid into a cavity of the inflatable member 104. If implanted around the urethra, the expansion of the inflatable member 104 causes the urethra to become restricted, thereby reducing the risk of incontinence in patients. For example, the electronic pump device 106 is configured to move fluid to pressurize the inflatable cuff (e.g., the inflatable member 104), which constricts the urethra, thereby restricting the flow of urine. To urinate, the patient may operate the electronic pump device 106 to depressurize the inflatable cuff by transferring fluid from the inflatable cuff to the fluid reservoir 102. If implanted into the corpus cavernosum, upon injection of the fluid into the inflatable member 104, the inflatable member 104 may increase its length and/or width, as well as increase its rigidity.

The fluid reservoir 102 may include a container having an internal chamber configured to hold or house fluid that is used to inflate the inflatable member 104. In some examples, the fluid reservoir 102 is pressurized. In some examples, the fluid reservoir 102 is a pressurized balloon. In some examples, the inflatable medical device 100 includes a single pressurized balloon. In some examples, the inflatable medical device 100 includes two or more pressurized balloons. The pressure in the inflatable member 104 may be generated by the fluid reservoir 102.

The inflatable medical device 100 may include a first tube member 103 and a second tube member 105. In some examples, the first tube member 103 and the second tube member 105 are referred to as conduit connectors. Each of the first tube member 103 and the second tube member 105 may define a lumen configured to transfer the fluid to and from the electronic pump device 106. The first tube member 103 may be coupled to the electronic pump device 106 and the fluid reservoir 102 such that fluid can be transferred between the electronic pump device 106 and the fluid reservoir 102 via the first tube member 103. For example, the first tube member 103 may define a first lumen configured to transfer fluid between the electronic pump device 106 and the fluid reservoir 102. The first tube member 103 may include a single or multiple tube members for transferring the fluid between the electronic pump device 106 and the fluid reservoir 102. In some examples, the first tube member 103 may be referred to as first tube members, and two first tube members can be connected together using a connector.

The second tube member 105 may be coupled to the electronic pump device 106 and the inflatable member 104 such that fluid can be transferred between the electronic pump device 106 and the inflatable member 104 via the second tube member 105. For example, the second tube member 105 may define a second lumen configured to transfer fluid between the electronic pump device 106 and the inflatable member 104. The second tube member 105 may include a single or multiple tube members for transferring the fluid between the electronic pump device 106 and the inflatable member 104. In some examples, the second tube member 105 may be referred to as second tube members, and two second tube members can be connected together using a connector. In some examples, the first tube member 103 and the second tube member 105 may include a silicone rubber material. In some examples, the electronic pump device 106 may be directly connected to the fluid reservoir 102. In some examples, the electronic pump device 106 may be directly connected to the inflatable member 104.

The electronic pump device 106 that can monitor control and regulate the pressure 116 within an inflatable member 104. In some examples, the electronic pump device 106 is referred to as a can. The electronic pump device 106 may automatically transfer fluid between the fluid reservoir 102 and the inflatable member 104 without the user manually operating a pump (e.g., squeezing and releasing a pump bulb). The electronic pump device 106 includes one or more processors 107 and one or more memory devices 109. The memory device(s) 109 may store executable instructions that when executed by the processor(s) 107 to execute operations discussed herein with respect to the electronic pump device 106. The memory device(s) 109 may store firmware 110. In some examples, the firmware 110 may be an operating system of the electronic pump device 106.

The electronic pump device 106 includes fluidic components 117 configured to enable the transfer of fluid between the inflatable member 104 and the fluid reservoir 102. The fluidic components 117 may include one or more pumps (e.g., electronic pumps, e.g., pumps operated by electrical signals) and one or more fluid valves. The electronic pump device 106 includes one or more pressure sensors 119 configured to generate pressure readings about a pressure 116 in the inflatable member 104 and/or a pressure in the fluid reservoir 102. In some examples, the pressure sensor 119 includes a first pressure sensor connected to the inflatable member 104, which generates pressure readings about the pressure 116 in the inflatable member 104 according to a sampling rate. In some examples, the pressure sensor 119 includes a second pressure sensor connected to the fluid reservoir 102, which generates pressure readings about the pressure in the fluid reservoir 102 according to a sampling rate.

The electronic pump device 106 includes an antenna 188 configured to wirelessly transmit (and receive) wireless signals from a controller 138. The controller 138 may be any type of component that can communicate with the electronic pump device 106. The controller 138 may be a computer, smartphone, tablet, pendant, wearable device (e.g., smartwatch, wristband, etc.), key fob, etc. A user may use the controller 138 to control the inflation and deflation of the inflatable member 104.

As shown in FIG. 1B, the controller 138 may include a patient application 140a executable by an external device 101a (also referred to as a user or a patient device). In some examples, the patient application 140a may perform any of the operations (and/or include any of the functionality) explained with reference to the clinician application 140b (and vice versa). The external device 101a may be any type of device having one or more processors 111a, one or more memory devices 113a, and an operating system 115a. The external device 101a may be a smartphone, a wearable device, or a tablet, etc. The memory device(s) 113a may store executable instructions that cause the external device 101a (e.g., the patient application 140a) to execute operations discussed herein.

In some examples, the electronic pump device 106 communicates with the external device 101a over a network 150, which may be a Wi-Fi connection, a mobile network connection, or a short-range communication network such as Bluetooth. In some examples, the electronic pump device 106 can communicate directly with a provider platform 152 executable by one or more server computers 160. The server computer(s) 160 includes one or more processors 161 and one or more memory devices 163. The memory device(s) 163 may store executable instructions that cause the processor(s) 161 to execute operations with respect to the provider platform 152 discussed herein. In some examples, the electronic pump device 106 and the external device 101a communicate with each other via a short-range communication network, and the external device 101a and the provider platform 152 communicate with each other via a Wi-Fi or mobile network communication network.

The patient application 140a is configured to communicate with the electronic pump device 106 and/or the provider platform 152. In some examples, the patient application 140a is installed on an operating system 115a of the external device 101a. In some examples, the patient application 140a is the operating system 115a or a sub-component of the operating system 115a. In some examples, the patient application 140a is a mobile (native) application. In some examples, the patient application 140a is a web application executable by a browser application. In some examples, the patient application 140a is a website of the provider platform 152.

The patient application 140a is configured to display a user interface 148a on a display 144a of the external device 101a. In some examples, the patient application 140a is associated with a user account 157a. In some examples, the user account 157a may store information about the patient and/or the inflatable medical device 100 such as patient data 180 and/or device data 182. In some examples, the user account 157a may include settings 139 of the inflatable medical device 100. In some examples, the user account 157a may include collected data 114 such as charging data and/or usage data. In some examples, information (or a portion thereof) of the user account 157a may be stored at the provider platform 152 and/or the electronic pump device 106. The charging data may be information about the user's charging activity, such as the frequency of charging, the charging levels, etc. In some examples, the usage data may include information about the use of the inflatable medical device 100 such as the frequency of inflate and/or deflate states, the duration of inflate and/or deflate states, and/or the pressure levels during the inflate or deflate states (e.g., including the target inflated pressure value 120, the target deflated pressure value 124).

The user interface 148a includes device controls 135 for controlling the inflation and deflation of the inflatable member 104. In some examples, the device controls 135 are UI elements. In response to selection of a first device control, the patient application 140a may transmit an inflate signal 136-1 to the electronic pump device 106. The electronic pump device 106 receives, via the antenna 188, the inflate signal 136-1, which causes the electronic pump device 106 to inflate the inflatable member 104 by activating one or more pumps and/or one or more valves, thereby transferring fluid from the fluid reservoir 102 to the inflatable member 104.

In response to selection of a second device control, the patient application 140a may transmit a deflate signal 136-2 to the electronic pump device 106. The electronic pump device 106 receives, via the antenna 188, the deflate signal 136-2, which causes the electronic pump device 106 to deflate the inflatable member 104 by activating one or more pumps and/or one or more valves, thereby transferring fluid from the inflatable member 104 to the fluid reservoir 102.

The patient application 140a may enable the user to select or adjust one or more settings 139 associated with the inflatable medical device 100. The settings 139 may include target inflated pressure value 120, the target deflated pressure value 124, pump rate, and/or other settings that can control the operation of the inflatable medical device 100. The patient application 140a may display one or more settings 139 on the user interface 148a. In some examples, when a setting 139 is adjusted, the patient application 140a may communicate with the electronic pump device 106 to update the setting 139 at the electronic pump device 106 and/or the provider platform 152.

The electronic pump device 106 may receive a target inflated pressure value 120 and/or a target deflated pressure value 124. A target inflated pressure value 120 may be a target pressure level of an inflate state. A target deflated pressure value 124 may be a target pressure level of a deflate state. For example, when the inflate state is activated, the electronic pump device 106 transfers fluid from the fluid reservoir 102 to the inflatable member 104 until the pressure 116 of the inflatable member 104 achieves the target inflated pressure value 120. When the deflate state is activated, the electronic pump device 106 transfers fluid from the inflatable member 104 to the fluid reservoir 102 until the pressure 116 of the inflatable member 104 achieves the target deflated pressure value 124. The electronic pump device 106 uses the pressure readings from the pressure sensor 119 to activate or deactivate the fluidic components 117 (e.g., one or more pumps and one or more valves) to transfer the fluid between the fluid reservoir 102 and the inflatable member 104 to achieve the target inflated pressure value 120 or the target deflated pressure value 124.

The electronic pump device 106 may receive the target inflated pressure value 120 and the target deflated pressure value 124 from the memory device 109 of the electronic pump device 106. The target inflated pressure value 120 and the target deflated pressure value 124 may be settings 139 that can be defined by a user (also referred to as a patient) and/or a clinician (also referred to as a physician). In some examples, the settings 139 may include an inflation delay value, e.g., a time delay between selecting to change the inflation pressure and the action of transferring fluid. During the inflated state or the deflated state, the member's pressure 116 may deviate from the target inflated pressure value 120 or the target deflated pressure value 124 by external forces, body movement, leakage from individual components of the electronic pump device 106, inadvertent over-inflation or under-inflation, and/or a user's pressure adjustments (e.g., manipulation) during the inflate state or the deflate state.

The electronic pump device 106 is configured to implement a pressure control system (e.g., a pressure controller 118) that can automatically activate the pump(s) to inflate or deflate such that the pressure 116 of the inflatable member 104 achieves the target inflated pressure value 120 and/or the target deflated pressure value 124. As shown in FIG. 1E, in response to the inflate signal 136-1 to initiate an inflate state, the electronic pump device 106 inflates the inflatable member 104 to the target inflated pressure value 120. The electronic pump device 106 determines a threshold level 122-1 (e.g., an upper threshold) and a threshold level 122-2 (e.g., a lower threshold) for the inflate state based on the target inflated pressure value 120. For example, the electronic pump device 106 may set the threshold level 122-1 as a pressure value (e.g., +X %) that is greater than the target inflated pressure value 120, and the threshold level 122-2 as a pressure value (e.g., −X %) that is less than the target inflated pressure value 120. In some examples, the threshold level 122-1 and the threshold level 122-2 are different from each other (e.g., the threshold level 122-1 is X % and the threshold level 122-2 is Y %).

In some examples, during the inflate state, a user may use a device control 135 (or a voice command 185) to adjust the pressure 116 of the inflatable member 104. In response to the new pressure set point (e.g., due to user manipulation) being different from the target deflated pressure value 124 by a threshold level, the electronic pump device 106 may update the target inflated pressure value 120 as the new pressure set point, and then re-determine the threshold level 122-1 and the threshold level 122-2 based on the updated target inflated pressure value 120.

In response to the pressure 116 of the inflatable member 104 during the inflate state being equal to or greater than the threshold level 122-1 for a period of time, the electronic pump device 106 may activate one or more pumps to transfer fluid from the inflatable member 104 to the fluid reservoir 102 until the pressure 116 achieves the target inflated pressure value 120 or is between the threshold level 122-1 and the threshold level 122-2, as shown in FIG. 1E (e.g. corrective inflation). In response to the pressure 116 of the inflatable member 104 during the inflate state being equal to or less than the threshold level 122-2, the electronic pump device 106 may activate one or more pumps to transfer fluid from the fluid reservoir 102 to the inflatable member 104 until the pressure 116 achieves the target inflated pressure value 120 or is between the threshold level 122-2 and the threshold level 122-1. In response to the pressure 116 of the inflatable member 104 during the inflate state being detected as being between the threshold level 122-1 and the threshold level 122-2, the pump(s) are deactivated, and the valve(s) are closed.

In response to a deflate signal 136-2 to initiate a deflate state, the electronic pump device 106 deflates the inflatable member 104 to the target deflated pressure value 124, as shown in FIG. 1E. The electronic pump device 106 determines a threshold level 126-1 (e.g., an upper threshold) and a threshold level 126-2 (e.g., a lower threshold) for the deflate state based on the target deflated pressure value 124. For example, during the deflated state, the electronic pump device 106 may set the threshold level 126-1 as a pressure value (e.g., +X %) that is greater than the target deflated pressure value 124, and the threshold level 126-2 as a pressure value (e.g., −X %) that is less than the target deflated pressure value 124. In some examples, the threshold level 126-1 and the threshold level 126-2 are different from each other (e.g., the threshold level 126-1 is X % and the threshold level 126-2 is Y %).

In some examples, during the deflate state, a user may use a device control 135 (or a voice command 185) to adjust the pressure 116 of the inflatable member 104. In response to the new pressure set point (e.g., due to user manipulation) being different than the target deflated pressure value 124 by a threshold level, the electronic pump device 106 updates the target deflated pressure value 124 as the new pressure set point, and then re-determine the threshold level 126-1 and the threshold level 126-2 based on the updated target deflated pressure value 124.

In response to the pressure 116 of the inflatable member 104 during the deflate state being equal to or greater than the threshold level 126-1 for a period of time, the electronic pump device 106 may activate one or more pumps to transfer fluid from the inflatable member 104 to the fluid reservoir 102 until the pressure 116 achieves the target deflated pressure value 124 or is between the threshold level 126-1 and the threshold level 126-2, as shown in FIG. 1E (e.g., corrective deflation). In response to the pressure 116 of the inflatable member 104 during the deflate state being equal to or less than the threshold level 126-2, the electronic pump device 106 may activate one or more pumps to transfer fluid from the fluid reservoir 102 to the inflatable member 104 until the pressure 116 achieves the target deflated pressure value 124 or is between the threshold level 126-2 and the threshold level 126-1. In response to the pressure 116 of the inflatable member 104 during the deflate state being detected as being between the threshold level 126-1 and the threshold level 126-2, the pump(s) are deactivated, and the valve(s) are closed.

In some examples, the electronic pump device 106 is configured to detect a forced deflate event 128 in the inflate state, and, in response to the forced deflate event 128, the electronic pump device 106 may initiate deflation of the inflatable member 104, e.g., to a target deflated pressure value 124 or to a lower pressure state. In some examples, when the forced deflate event 128 is detected or triggered, the electronic pump device 106 causes the valves to open to relieve the pressure 116 to a lower state. In some examples, the forced deflate event 128 includes detection of a disconnection to a wireless connection between the controller 138 and the electronic pump device 106. In some examples, the controller 138 and the electronic pump device 106 are wirelessly connected (e.g., paired), and when the electronic pump device 106 detects that the electronic pump device 106 is not wirelessly connected to the external device 101a during the inflate state, the electronic pump device 106 may automatically cause the inflatable member 104 to be disconnected.

In some examples, the forced deflate event 128 includes the detection of the pressure 116 of the inflatable member 104 being equal to or greater than a maximum threshold in the inflate state. While in the inflate state, the electronic pump device 106 may detect whether the pressure 116 of the inflatable member 104 is equal to or greater than the maximum threshold, and, if so, the electronic pump device 106 automatically initiates a deflate state. The maximum threshold is a maximum pressure threshold of the inflatable member 104, where, when the pressure 116 exceeds that value, the electronic pump device 106 automatically causes the inflatable member 104 to deflate.

In some examples, the forced deflate event 128 may be triggered by a pressure 116 that presents potential damage to the device. If the pressure 116 exceeds this threshold, then deflation is initiated. In some examples, the forced deflate event 128 may be triggered by a pressure 116 that presents harm to the patient or damage to patient tissue. The forced deflate event 128 may be triggered by a combination of pressure and time. For example, if the device is inflated for a set amount of time without it being deflated, the device may automatically deflate to prevent harm to the patient. In some examples, the forced deflate event 128 may be triggered in response to detection of a sudden change in pressure 116 (e.g., a steady reading of X (e.g., 10 psi) increasing to a steady reading of Y (e.g., 15 psi) may force a deflation in order to calibrate the device to ensure it functions properly and prevent the use of the device in an unknown or improperly controlled (e.g., pressure control) state).

In some examples, the forced deflate event 128 may be triggered in response to the detection of a magnetic field being applied to the device, e.g., to force a deflate in case of malfunction. In some examples, the forced deflate event 128 may also be triggered in response to the detection of a flow of current from a charger, e.g., the current potentially being used to charge the battery 112. In some examples, the forced deflate event 128 may be triggered in response to the detection of a battery level being less than a threshold level (e.g., the battery 112 is too low to inflate and/or deflate the device). For example, when a voltage threshold on the battery 112 is detected, the forced deflate event 128 may be triggered. Forced deflate event 128 could also be programed by a physician or patient that after a certain time it will deflate. This could be to remove the need to find the controller 138 to activate a deflation or to prevent the device from remaining inflated for too long of a period of time. In some examples, the forced deflate event 128 may be triggered in response to the detection of a loss of a pressure signal (e.g., as the control of the device could be impaired or lost).

In some examples, the electronic pump device 106 operates in one of a plurality of operating modes 134. As shown in FIG. 1F, the operating modes 134 may include a storage mode 134-1, an end-of-life mode 134-2, an active mode 134-3, and a disconnected mode 134-4. In some operating modes 134, certain features or capabilities may be limited. In some examples, at least a portion of the inflatable medical device 100 may be included in a medical device package. For example, the inflatable member 104, the fluid reservoir 102, the electronic pump device 106, and the tube members 103, 105 may be included in the medical device package. In some examples, the electronic pump device 106 is in the storage mode 134-1 when in the medical device package.

An electronic pump device 106 in the storage mode 134-1 may disable wireless communications and operations of a pump and valve, etc. In some examples, in response to the detection of a magnetic field, the electronic pump device 106 changes the operating mode 134 of the electronic pump device 106 from the storage mode 134-1 (e.g., to the active mode 134-3 or the disconnected mode 134-4). For example, the electronic pump device 106 is configured to detect the presence of a magnetic field. For example, a person may place a charger device 184 or a magnet in proximity to the electronic pump device 106. The charger device 184 may be used to charge a battery 112 of the electronic pump device 106 while the device is in the body of the patient but may also be used to switch the operating mode 134 from the storage mode 134-1. A charger device 184 may use wireless charging (e.g., inductive charging, capacitive charging) that transfers electromagnetic energy from a transmitter coil in the charger device 184 to a receiver coil in the electronic pump device 106. For example, a user may hold the charger device 184 to a position on the user's body (outside of the body) that generally aligns with a position of the electronic pump device 106 that is located inside of the body.

In response to the detection of the presence of a magnetic field, the electronic pump device 106 may switch the operating mode from the storage mode 134-1 to another operating mode 134 such as an active mode 134-3 or a disconnected mode 134-4. In some examples, the magnetic field will induce the flow of current, e.g., to start charging the battery 112. In some examples, the detection of the flow of current (e.g., start of the charging of the battery 112) may switch the device operating mode from the storage mode 134-1 to the active mode 134-3. The active mode 134-3 may be an operating mode 134 in which device's functions are not disabled or restricted. The disconnected mode 134-4 is an operating mode 134 that disables inflating the inflatable member 104. In some examples, the electronic pump device 106 is placed in the disconnected mode 134-4 when the controller 138 and the electronic pump device 106 are disconnected.

The end-of-life mode 134-2 is an operating mode 134 of the electronic pump device 106 that may implement one or more actions (e.g., initiation of a notification(s) or message(s) to the patient and/or clinician) and/or restrict one or more functions of the electronic pump device 106. In some examples, when the end-of-life mode 134-2 is enabled on the electronic pump device 106, the electronic pump device 106 may initiate a notification that is displayed on the external device 101a, which indicates that the device is nearing its end of life, and to schedule a replacement procedure. In some examples, when the end-of-life mode 134-2 is enabled on the electronic pump device 106, inflating the inflatable member 104 is disabled. In some examples, the electronic pump device 106 may initiate a timer 133 (e.g., a countdown timer), and, in response to the expiration of a time instance programmed by the timer 133, the electronic pump device 106 may switch the operating mode 134 to the end-of-life mode 134-2. In some examples, the timer 133 is initiated when the electronic pump device 106 is taken out of the storage mode 134-1.

In some examples, the controller 138 includes a clinician application 140b executable by an external device 101b, as shown in FIG. 1C. The clinician application 140b is configured to display a user interface 148a on a display 144a of the external device 101b. In some examples, the clinician application 140b may enable a clinician to set one or more settings 139, view collected data 114 (e.g., usage data, charging data), and/or control the electronic pump device 106 using device controls 135. In some examples, the clinician application 140b may receive, via the user interface 148a, a clinician-defined setting (e.g., a setting 139), and transmit the clinician-defined setting to the electronic pump device 106, which is stored in the memory device 109. For example, a clinician may use the clinician application 140b to set certain settings 139 for the electronic pump device 106. In some examples, the clinician application 140b may receive patient data 180 and/or device data 182 (e.g., the clinician may enter this information), which may be stored on the provider platform 152 and/or the electronic pump device 106.

In some examples, the clinician can use the clinician application 140b to scan a barcode of the inflatable medical device 100, which can automatically populate the user interface 148b with the device data 182. For example, the clinician application 140b may enable the external device's camera to scan a barcode, which may identify the inflatable medical device 100 from a database 155. In some examples, the clinician application 140b may provide the physician (or other health care provider or sales representative present during the surgery and/or providing support) the ability to enter pertinent information regarding the patient and/or implanted product. In some examples, the clinician application 140b may transmit the patient data 180 and the device data 182 to the electronic pump device 106 for storage in the memory device 109. In some examples, the clinician may use the clinician application 140b to initiate the creation of a user account 157a at the provider platform 152, and the patient data 180 and the device data 182 may be stored in the user account 157a at the database 155. In some examples, the patient data 180 and the device data 182 are stored at the memory device 109 of the electronic pump device 106. The patient data 180 may include personal information such as name, birth date, age, and/or other information about the patient. The device data 182 may identify the type of inflatable medical device 100, including cylinder size and type.

The external device 101b may be an example of the external device 101a and may include any of the details discussed with reference to the external device 101a. The external device 101b includes one or more processors 111b, one or more memory devices 113b, and an operating system 115b. The clinician application 140b may be associated with a user account 157b, which may also be stored on provider platform 152. The clinician application 140b may be an example of the patient application 140a and may include any of the details discussed with reference to the patient application 140a.

The clinician application 140b may render a user interface 148b on a display 144b of the external device 101b. In some examples, the patient application 140a and the clinician application 140b are different versions of a client application configured to communicate with the provider platform 152 and the electronic pump device 106. In some examples, the clinician application 140b includes different or additional features as compared to the patient application 140a. For example, a clinician may use the clinician application 140b to create a user account 157a for a patient, enter patient data 180 and/or device data 182, determine or adjust one or more settings 139, and/or view certain information, which may not be available with the patient application 140a.

In some examples, the clinician's external device 101b is a managed device by a device management engine 156 of the provider platform 152. In some examples, the device management engine 156 is a mobile device management system configured to manage external devices 101b. For example, the device management engine 156 may enable an administrator (e.g., using a provider device 101c) to configure, manage, monitor, and/or secure a clinician's external device 101b. For example, the device management engine 156 may manage the clinician application 140b, the operating system 115b, or other applications 140b on the external device 101b.

The clinician's external device 101b includes a management agent 186 configured to receive enforcement policies 132 from the provider platform 152 and apply the enforcement policies 132 to manage computer functions of the external device 101b. The management agent 186 may control the installation of applications, implement security policies, manage notifications, and/or access or deny certain web content.

In some examples, the inflatable medical device 100 includes a provider application 140c executable by a provider device 101c. The provider application 140c can render a user interface 148c on a display 144c of the provider device 101c. The provider device 101c may be an example of the external device 101a or the external device 101b and may include any of the details discussed with reference to the external device 101a or the external device 101b. The provider device 101c includes one or more processors 111c, one or more memory devices 113c, and an operating system 115c. The provider application 140c may provide an operation interface 190 with the ability for an administrator to deploy software updates 154 (e.g., new device firmware 110, update to the device firmware 110, security updates, etc.) to the electronic pump device 106, the patient application 140a, and/or the clinician application 140b.

In some examples, a software update 154 can be applied to the device's firmware 110 while the electronic pump device 106 is implanted in the user's body. In some examples, an administrator may use the operation interface 190 to upload a software update 154 to the provider platform 152. The provider platform 152 may transmit the software update 154 to the electronic pump device 106, and the electronic pump device 106 may update the firmware 110 with the software update 154. In some examples, when a software update 154 is available on the provider platform 152, the patient application 140a (or the clinician application 140b) may retrieve, over the network 150, the software update 154. When the patient application 140a (or the clinician application 140b) is connected to the electronic pump device 106, the patient application 140a (or the clinician application 140b) may transmit the software update 154 to the electronic pump device 106 for installation on the electronic pump device 106. For example, the electronic pump device 106 may receive, via the antenna, the software update 154 from the external device 101a (e.g., the patient application 140a) and update the firmware 110 using the software update 154.

In some examples, the inflatable medical device 100 includes a customer support interface 192 with one or more deactivate controls 194 that enable an administrator to deactivate a patient application 140a and/or a clinician application 140b. In some examples, the customer support interface 192 may be used to enter registration information 196 on the provider platform 152. The registration information 196 includes such as patient and equipment enrollment information on the provider platform and/or registering new clinician applications and/or clinician devices.

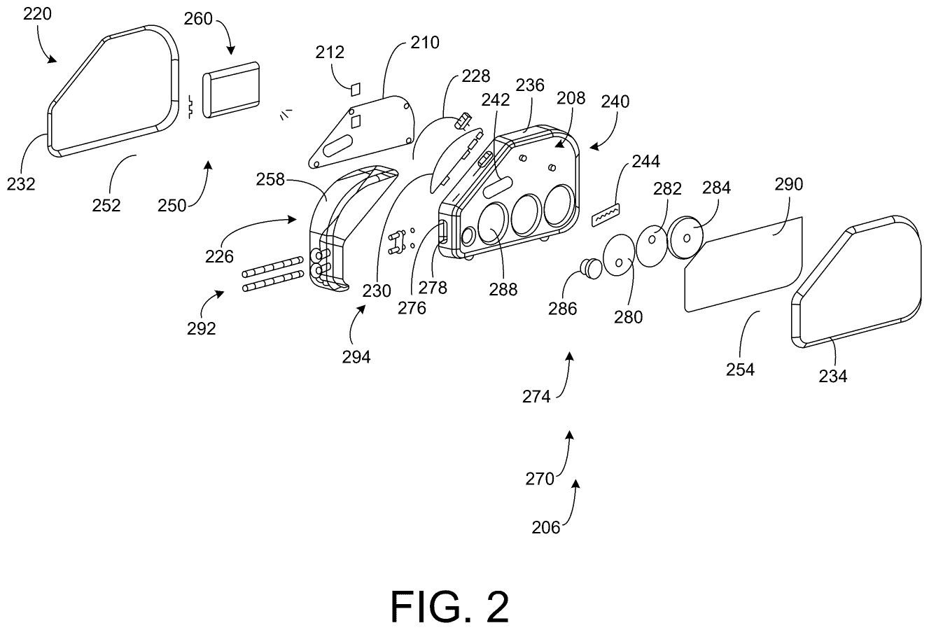

FIG. 2 illustrates an example of an exploded view of an electronic pump device 206 according to an aspect. The electronic pump device 206 may be an example of the electronic pump device 106 of FIGS. 1A to 1F and may include any of the details discussed with reference to FIGS. 1A to 1F. The electronic pump device 206 includes a housing 220 with a first sidewall 232, a second sidewall 234, a peripheral wall 236, and a frame 240. The first sidewall 232, second sidewall 234, and the peripheral wall 236 are hermetically sealed together to form an internal compartment 250 within the housing 220.

The frame 240 is disposed within the internal compartment 250 to form a first partition 252 and a second partition 254 in such a manner that the first partition 252 is hermetically sealed from the second partition 254. The frame 240 can be integrally formed with the peripheral wall 236, the first sidewall 232, and/or the second sidewall 234. In some examples, the frame 240 is welded to the peripheral wall 236 or welded to the first sidewall 232, and/or the second sidewall 234. The first sidewall 232, the peripheral wall 236, and the frame 240 may form the first partition 252. The second sidewall 234, the peripheral wall 236, and the frame 240 may form the second partition 254, which is opposite the frame 240 from the first partition 252.

The electronic pump device 206 can include a header 226 attached to the housing 220 to form an internal region 258 between an inner surface of the header 226 and an outer surface of the housing 220 that includes power and communication interface structures such as a secondary coil 228 and the antenna 230 external to the hermetically sealed housing 220. The header 226 is configured from a dielectric or insulative material, such as a radome, to allow the transmission of power and communication signals between the antenna 230 and a handset programmer or charger, and between the secondary coil 228 and the charger. For example, the header 226 may include an over molded polymer affixed to the housing 220 and including the secondary coil 228 and the antenna 230 within the internal region 258. The secondary coil 228 and antenna 230 are constructed from a biocompatible material. In some examples, the secondary coil 228 and antenna 230 can be formed as a coil from a stamped titanium core clad with gold or silver. In some examples, the secondary coil 228 and antenna 230 can be formed from a gold wire.

The electronic pump device 206 includes an energy storage system, such as a battery (e.g., a rechargeable power source) (e.g., a rechargeable battery), and electronic components 212 within the first partition 252. The electronic components 212 can be disposed on a circuit substrate 210, such as a plurality of circuit boards, within the first partition 252. The battery 260 can assume various forms appropriate to provide power for generating desired electrical signals and to store power provided from the electronic components 212. For example, the battery 260 can incorporate lithium-ion (Li+) chemistry, e.g., a lithium-ion battery to operate the electronic components 212. In some examples, the electronic components 212 can be implemented by various components including resistors, capacitors, transistors, and integrated circuits disposed on the circuit substrate 210. The secondary coil 228 and antenna 230 are electrically coupled to the electronic components 212 within the first partition 252, such as via a hermetic feedthrough component.

The electronic components 212 can include a recharge system, a communication system, and a controller. The recharge system includes hardware configured to interface with the secondary coil 228 to receive power signals, and to provide the power signals in a form suitable to recharge the battery 260 and can include circuitry to reduce the likelihood of overcharging the battery 260. The communication system includes hardware configured to interface with the antenna 230 to receive electrical communication signals. For instance, the communication system can be configured to communicate via a wireless personal area network technology such as a short-range communication protocol (e.g., Bluetooth) (e.g., Bluetooth Low Energy), which is compatible with several operating systems that can be applied in mobile devices configured as external devices (e.g., handset programmers). The communication system can include an integrated circuit to implement an applied communication technology. In some examples, the communication system can be used to transmit communication signals to other devices, such as a charger or the handheld programmer (e.g., external device), and the communication system can be implemented to generate communication signals and provide the communication signals to the antenna 230 for transmission. In some examples, the communication system can be configured to receive and transmit radio frequency signals via the antenna 230. The controller can include a microcontroller to operate the recharge system and to receive and operate in response to communication signals or generate communication signals from the communication system.

The electronic pump device 206 also includes a fluidic circuit 270 within the second partition 254 and opposite the frame 240 from the battery 260 and electronic components 212. In some examples, the frame 240 can include an opening 242 that includes a hermetic interface 244, such as a feedthrough hermetically affixed to the frame 240. The electronic components 212 are operably coupled to the fluidic circuit 270 across the frame 240 via the hermetic interface 244. For example, the controller of the electronic components 212, powered by the battery 260, can cause the operation of the fluidic circuit 270 such as to control and monitor the fluidic circuit 270.

The fluidic circuit 270 includes a fluidic manifold 208 and fluidic components 274 operably coupled to the fluidic manifold 208. In some examples, the fluidic manifold 208 is a structure integrated into the frame 240 such that the fluidic manifold 208 and the frame 240 together form the hermetic barrier between the first partition 252 and the second partition 254 of the internal compartment 250. For instance, the battery 260, the circuit substrate 210, or electronic components 212 can be coupled to a first major surface of the fluidic manifold 208 in the first partition 252, and the fluidic components 274 are operably coupled to a second, and opposite major surface of the fluidic manifold 208 in the second partition 254.

The fluidic circuit 270 provides for the transfer of the fluid between the fluid reservoir (e.g., the fluid reservoir 102 of FIGS. 1A to 1C) and the inflatable member (e.g., the inflatable member 104 of FIGS. 1A to 1C). The fluidic manifold 208, which can be a hermetic manifold, segments and contains the fluid from the internal compartment 250 to reduce the chance of fluid exchange and directs the fluid from a first port 276 to a second port 278 via internal fluid passageways or channels.

The fluidic components 274 include a plurality of fluid pumps, such as pumps 280, 282, a valve 284 mounted into the fluidic manifold 208 in fluidic communication with a manifold passageway to transfer fluid from the first port 276 to the second port 278. The pumps and the valve(s) are in fluid communication with a single fluid passageway between ports 276, 278. The fluidic components 274 also includes one or more pressure sensors 286 operably coupled to the fluidic manifold 208 and in fluidic communication with the passageway to detect a pressure of the fluid within the fluidic manifold 208.

In some examples, the fluidic components 274 are included in a planar configuration on the fluidic manifold 208 in which the pumps 280, 282, valve 284, and pressure sensor 286 are mounted into the fluidic manifold 208 on a plane for slim profile within the second partition 254. The fluidic manifold 208 can include chambers 288 formed into the second major surface in which the chambers are fluidically coupled to the single passageway within the fluidic manifold 208. The chambers are configured to receive the pumps 280, 282, and valve 284 and one or more pressure sensors 286. In some examples, the fluidic manifold 208 can receive a piezoelectric pump. The fluidic manifold 208 can receive a component cover 290 over the fluidic components 274, which can be hermetically sealed to the second major surface.

In some examples, the electronic pump device 206 may include kink resistant tubing 292 that can extend through the header 226 and attach to the ports 276, 278 via components such as a barb 294 and O-rings. The kink resistant tubing 292 can be attached to the tube members (e.g., tube members 103, 105 of FIGS. 1A to 1C) to fluidically couple the electronic pump device 206 to the fluid reservoir and the inflatable member.

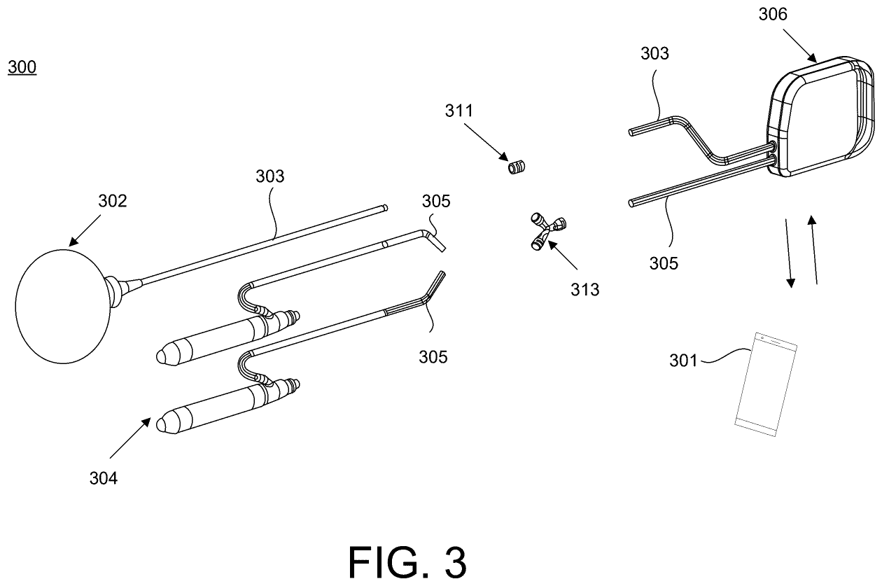

FIG. 3 illustrates a perspective of an inflatable penile prosthesis 300 according to an aspect. The inflatable penile prosthesis 300 may be an example of any of the medical devices discussed herein (e.g., including inflatable medical device 100), and, therefore, may include any of the details discussed with reference to the previous figures.

The inflatable penile prosthesis 300 includes an inflatable member 304, a fluid reservoir 302, and an electronic pump device 306. The inflatable member 304 includes a pair of inflatable cylinders. The electronic pump device 306 may be an example of any of the pump devices discussed with reference to the previous figures and may include any of the details discussed herein. The electronic pump device 306 includes fluidic components such as pumps, valves, and/or sensing devices positioned in fluid passageways. The electronic pump device 306 includes components such as, for example, one or more fluid control devices, one or more pressure sensors, and other such components. The electronic pump device 306 includes an electronic control system configured to provide for the transfer of fluid between a fluid reservoir 302 and an inflatable member 304 via the fluidic components.

The electronic pump device 306 may include one or more integrated circuits. In some examples, the integrated circuits are included in a printed circuit board that is included in a housing of the electronic pump device 306. Fluidic components and the electronic components of the electronic pump device 306 are included in a housing. In some examples, fluidic components and electronic components in the housing define a manifold (e.g., an electronically controlled fluidic manifold) that provides for the electronic control of the flow of fluid between the fluid reservoir 302 and the inflatable member 304. In some examples, the electronic pump device 306 can communicate with an external device 301, via respective communication modules. For example, an application stored in a memory and executed by a processor of the external device 301 may allow the user and/or a physician to operate, view, monitor and alter operation of the inflatable penile prosthesis 300. The external device 301 may be the external device 101a of FIGS. 1A to 1F or the external device 101b of FIGS. 1A to 1F.

The inflatable penile prosthesis 300 includes one or more first tube members 303 that connect a first fluid port of the electronic pump device 306 with the fluid reservoir 302. One or more second tube members 305 connect a second fluid port of the electronic pump device 306 with the inflatable member 304 in the form of the inflatable cylinders. In some examples, the inflatable penile prosthesis 300 includes a connector 311 that is used to connect two tube members 303 together, and a connector 313 that is used to connect two tube members 305 together.

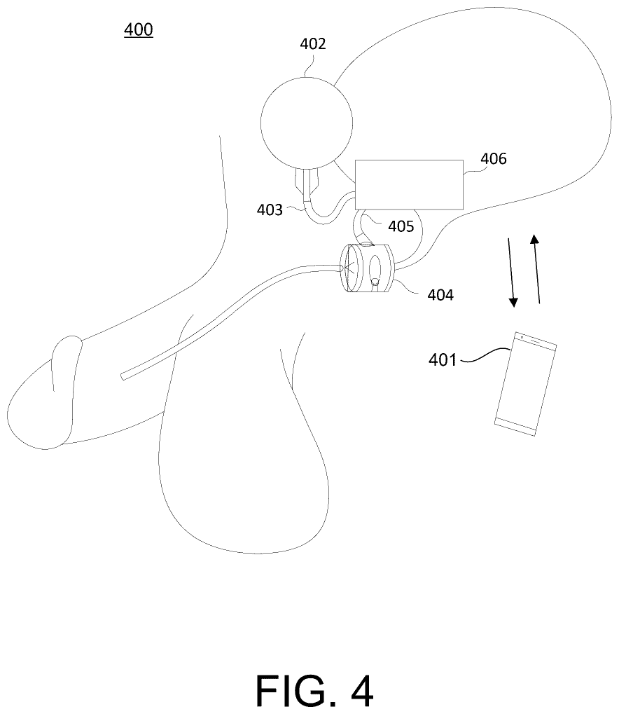

FIG. 4 illustrates a urinary control device 400 having an electronic pump device 406 according to an aspect. The urinary control device 400 may be an example of the inflatable medical device 100. In some examples, the urinary control device 400 is an artificial urinary sphincter device. The electronic pump device 406 may include any of the features of the pump devices discussed herein. The urinary control device 400 includes an electronic pump device 406, a fluid reservoir 402, and a cuff 404 (e.g., an inflatable cuff).

The fluid reservoir 402 may be a pressure-regulating inflation balloon or element. The fluid reservoir 402 is in operative fluid communication with the cuff 404 via one or more tube members 403, 405. The fluid reservoir 402 is constructed of polymer material that is capable of elastic deformation to reduce fluid volume within the fluid reservoir 402 and push fluid out of the fluid reservoir 402 and into the cuff 404. However, the material of the fluid reservoir 402 can be biased or include a shape memory construct adapted to generally maintain the fluid reservoir 402 in its expanded state with a relatively constant fluid volume and pressure. In some examples, this constant level of pressure exerted from the fluid reservoir 402 to the cuff 404 will keep the cuff 404 at a desired inflated state when open fluid communication is provided between the fluid reservoir 402 and the cuff 404. In some examples, the fluid reservoir 402 is implanted into the abdominal space.

A user may use an external device 401 to control the urinary control device 400. In some examples, the user may use the external device 401 to inflate or deflate the cuff 404. For example, in response to the user activating an inflation cycle using the external device 401, the external device 401 may transmit a wireless signal to the electronic pump device 406 to initiate the inflation cycle to transfer fluid from the fluid reservoir 402 to the cuff 404 (e.g., by opening an active valve where the pressure in the fluid reservoir 402 causes the fluid to move through the active valve to the cuff 404). In some examples, in response to the user activating a deflation cycle using the external device 401, the external device 401 may transmit a wireless signal to the pump device 406 to initiate the deflation cycle to transfer fluid from the cuff 404 to the fluid reservoir 402. The external device 401 may be the external device 101a of FIGS. 1A to 1F or the external device 101b of FIGS. 1A to 1F.

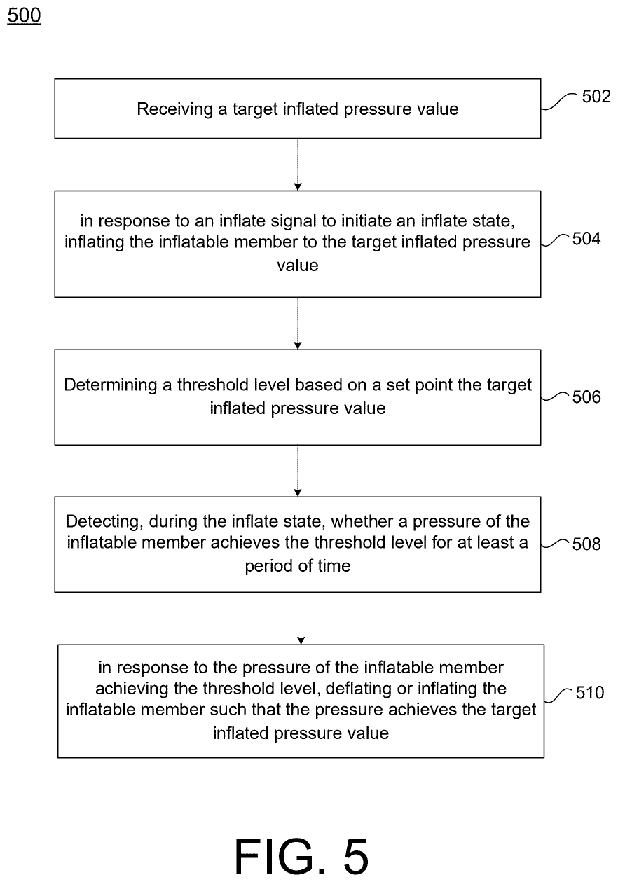

FIG. 5 illustrates a flowchart 500 depicting example operations of regulating pressure of an inflatable medical device. Although the flowchart 500 of FIG. 5 illustrates the operations in sequential order, it will be appreciated that this is merely an example, and that additional or alternative operations may be included. Further, operations of FIG. 5 and related operations may be executed in a different order than that shown, or in a parallel or overlapping fashion.

Operation 502 includes receiving a target inflated pressure value. Operation 504 includes, in response to an inflate signal to initiate an inflate state, inflating the inflatable member to the target inflated pressure value. Operation 506 includes determining a threshold level based on a set point the target inflated pressure value. Operation 508 includes detecting, during the inflate state, whether a pressure of the inflatable member achieves the threshold level for at least a period of time. Operation 510 includes, in response to the pressure of the inflatable member achieving the threshold level, deflating or inflating the inflatable member such that the pressure achieves the target inflated pressure value.

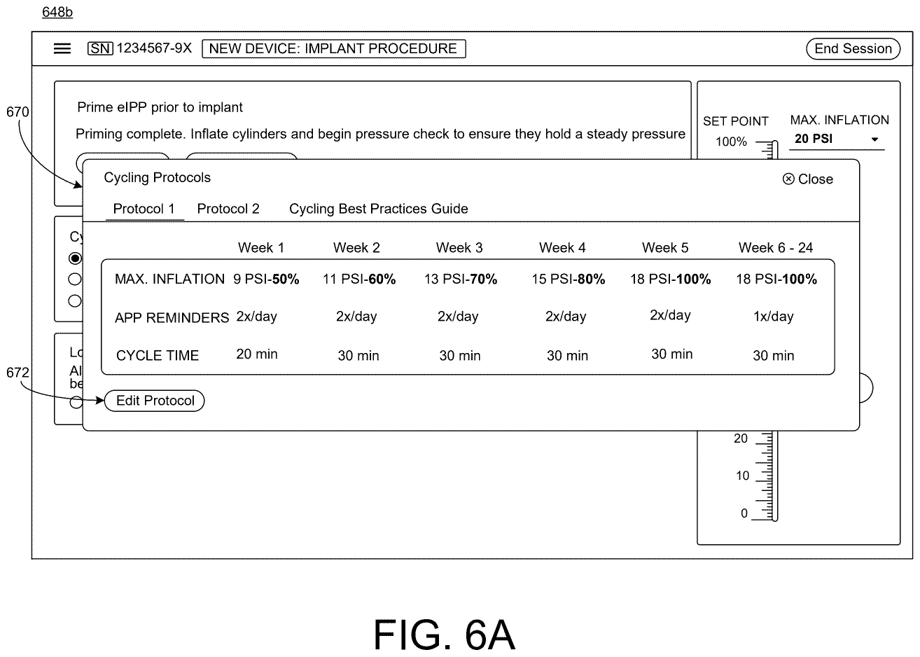

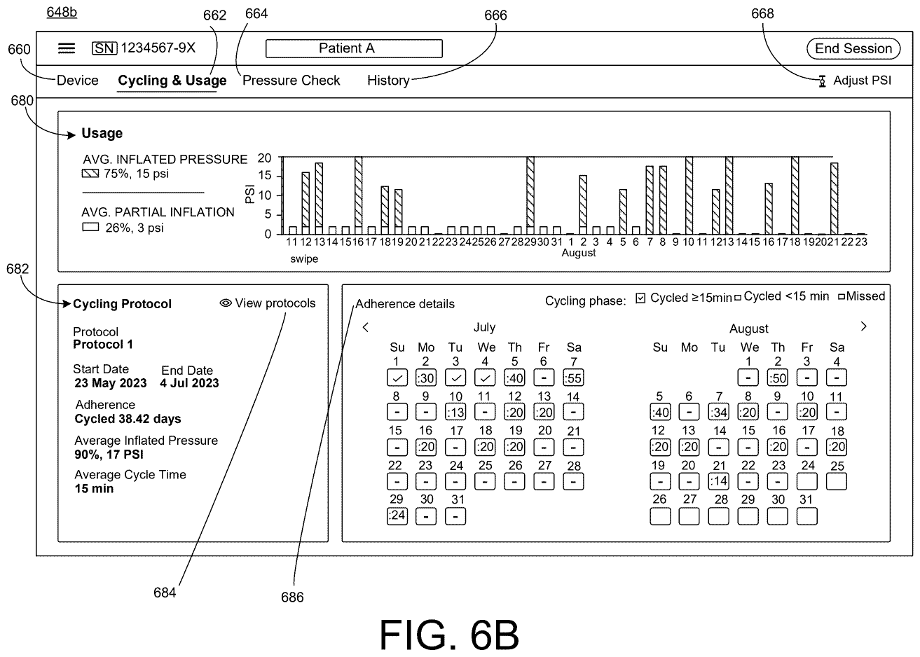

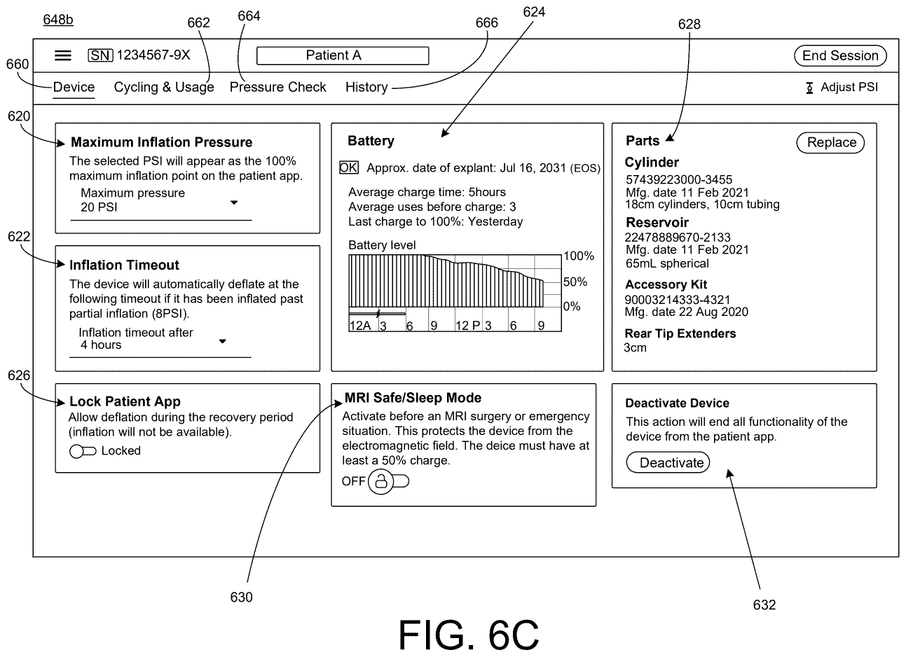

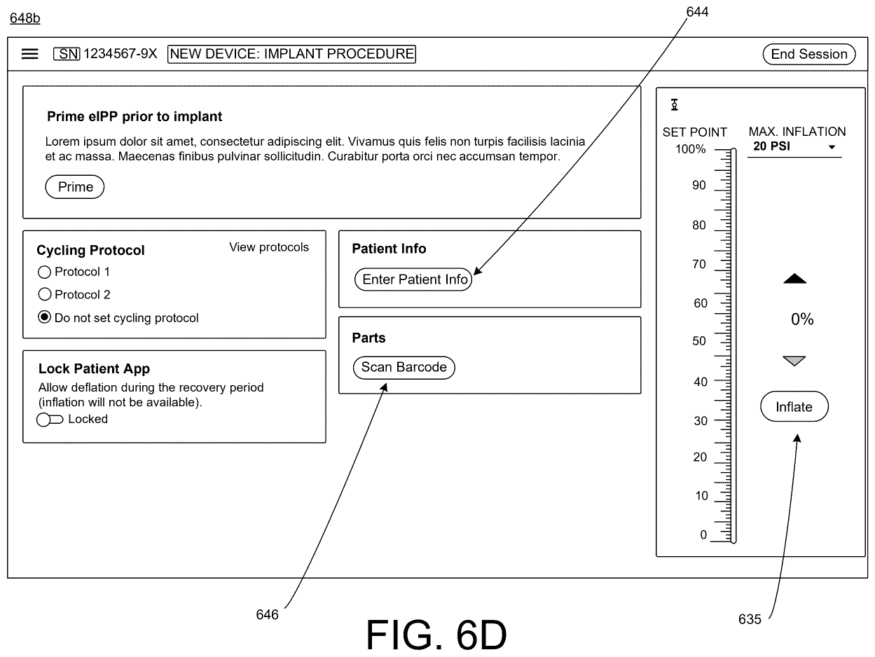

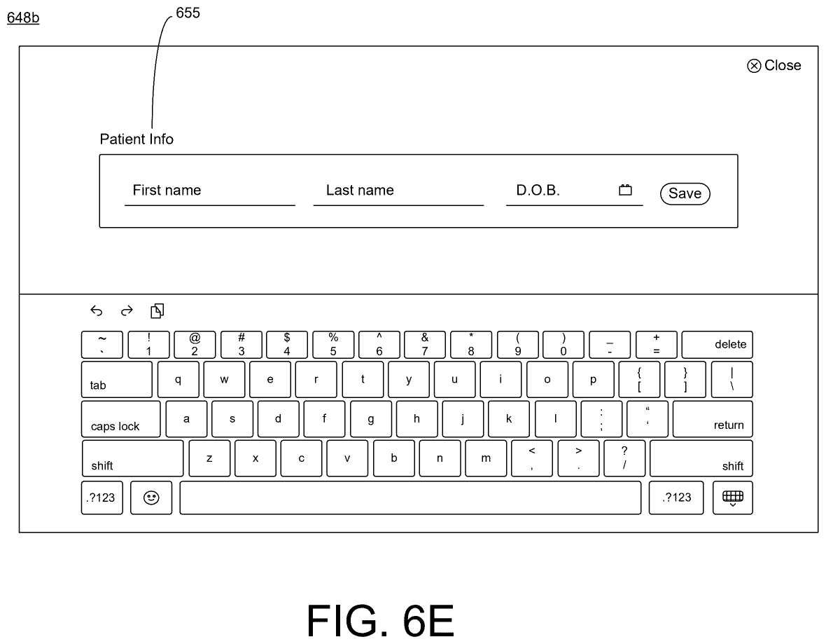

FIGS. 6A to 6E illustrate examples of a user interface 648b according to an aspect. In some examples, the user interface 648b is an example of an interface of the clinician application 140b of FIGS. 1A to 1F. In some examples, the user interface 648b is an example of an interface of the patient application 140a of FIGS. 1A to 1F. Referring to FIG. 6A, the user interface 648b depicts a computer object 670 with one or more cycling protocols for the implantable medical device 100 of FIGS. 1A to 1F. A cycling protocol may be a post-operative process for inflatable penile prosthesis. The cycling protocol involves inflating and deflating the device regularly (e.g., over time periods) to prevent the formation of scar tissue around the implant, which can restrict its movement and function.

For each time period (e.g., week), as shown in FIG. 6A, a cycling protocol may define a maximum inflation pressure, a number of application reminders, and a cycling time. In some examples, a clinician may use their clinician application to define one or more cycling protocols and select a particular cycling protocol. In some examples, the computer object 670 includes an edit control 672, which, when selected, causes the clinician application 140b to render an interface that allows the clinician to modify the maximum inflation pressure, application reminders, cycle time, and/or other settings associated with a particular cycling protocol.

In some examples, as shown in FIG. 6B, the user interface 648b includes a tabbed interface, e.g., with a device section 660, a cycling and usage section 662, a pressure check section 664, and a history section 666. The FIG. 6B depicts the cycling and usage section 662. As shown in FIG. 6B, the user interface 648b includes usage data 680. The usage data 680 may include information about the usage of the inflatable medical device. The usage data 680 may include one or more metrics and/or graphics that depict the usage of the inflatable medical device over time. In some examples, the usage data 680 includes an average inflatable pressure over time and an average partial inflation pressure over time. In some examples, the usage data 680 includes a graphic (e.g., a chart) that depicts the pressure of the inflatable medical device over time. In some examples, the user interface 648b includes a pressure adjust control 688, which, when selected, causes the clinician application to render an interface to edit one or more pressure levels as discussed with reference to FIGS. 1A to 1E.

The user interface 648b includes cycling protocol data 682 about a cycle protocol implemented by the inflatable medical device. The cycling protocol data 682 identifies a selected cycling protocol, a duration of the cycling protocol, adherence information about the patient's adherence to the cycling protocol, an average inflated pressure, and/or an average cycle time. In some examples, the user interface 648b includes a view protocol control 684, which, when selected, causes the clinician application to render an interface (e.g., FIG. 6A) to view the protocols. The user interface 648b includes adherence details 686 about the patient's adherence to the cycling protocol. The adherence details 686 may include a graphic that depicts whether a cycling phase is completed, not completed, or missed.

FIG. 6C illustrates the device section 660, which depicts information about the device. The device section 660 may depict one or more settings for the device. The clinician may use the clinician application to adjust one or more of the settings. The device section 660 includes a maximum information pressure 620 (e.g., a selected PSI will appear as the 100% maximum information on a patient app), an inflation timeout 622 (e.g., the device will automatically deflate at a specified timeout (e.g., four hours - which can be adjusted by the clinician) if it has been inflated past a partial inflation level), a lock patient app setting 626 (e.g., this allows deflation during the recovery period where inflation will not be available), battery data 624, an MRI safe mode setting 630, parts data 628, and/or a deactivate device control 632. The battery data 624 provides information about the battery such as average charge time, average uses before change, last charge to 100%, and/or battery levels over a period of time. The MRI safe mode setting 630, which, when activated, protects the device from an electromagnetic field. The parts data 628 includes information about the parts of the device such as cylinder, reservoir, accessory kit, and/or rear tip extenders. The deactivate device control 632, which, when selected, causes the clinician application to initiate deactivation of a patient application.

FIG. 6D illustrates a user interface 648b for registering a device with the system. As shown in FIG. 6D, the user interface 648b may include an enter patient information control 644, which, when selected, causes the clinician application to render the interface of FIG. 6E. In FIG. 6E, the user interface 648b includes an interface to enter patient data 655 into the system. As shown in FIG. 6D, the user interface 648b includes a scan bar code control 646, which, when selected, causes the clinician application to render an interface to accept a bar code of the parts of the device. In FIG. 6D, the user interface 648b includes a device control 635 that allows a clinician to set a maximum inflation with controls to inflate and/or deflate.

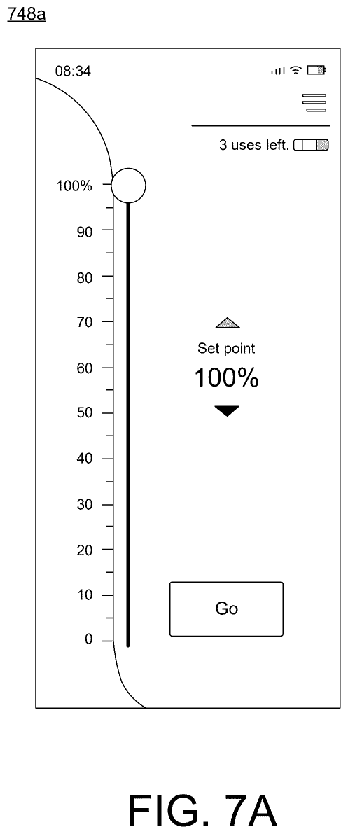

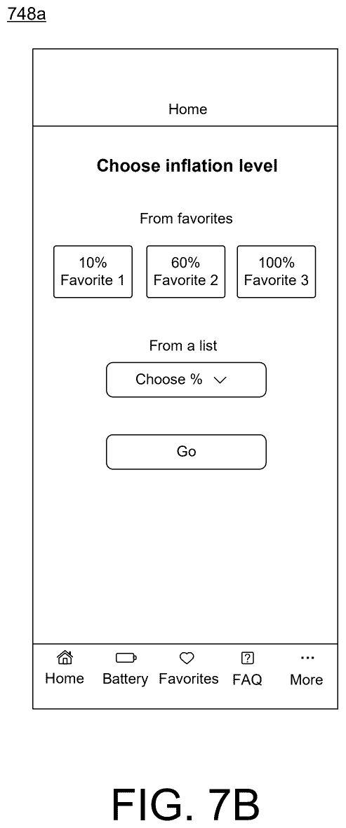

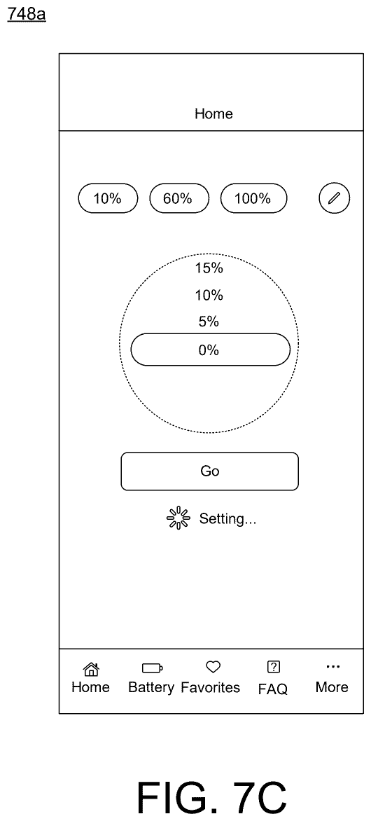

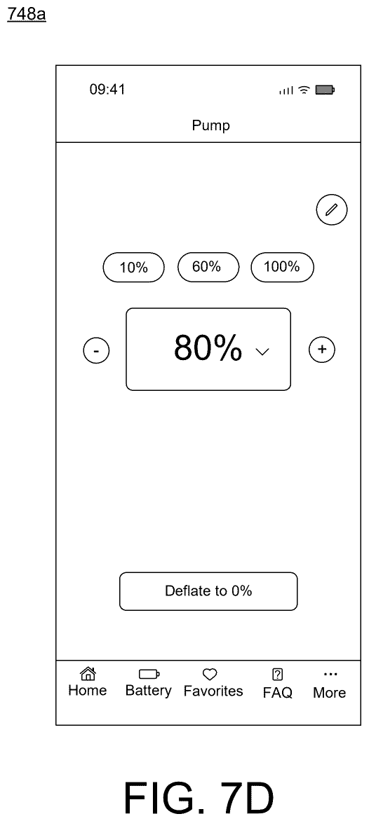

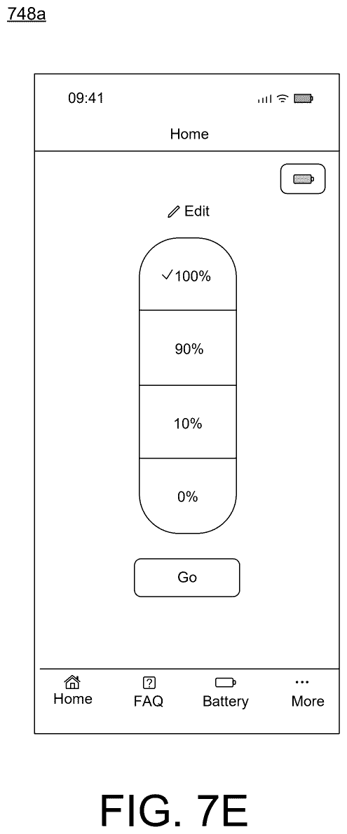

FIGS. 7A to 7E illustrate examples of a user interface 748a according to an aspect. In some examples, the user interface 748a is an example of an interface of the patient application 140a of FIGS. 1A to 1F. In some examples, the user interface 748a is an example of an interface of the clinician application 140b of FIGS. 1A to 1F. FIGS. 7A to 7E may depict various examples of a device control for selecting an inflation level.

Clause 1. An inflatable medical device comprising: an electronic pump device configured to transfer fluid between an inflatable member and a fluid reservoir, the electronic pump device configured to execute operations, the operations comprising: receiving a target inflated pressure value; in response to an inflate signal to initiate an inflate state, inflating the inflatable member to the target inflated pressure value; determining a threshold level based on the target inflated pressure value; detecting, during the inflate state, whether a pressure of the inflatable member achieves the threshold level for at least a period of time; and in response to the pressure of the inflatable member achieving the threshold level for at least the period of time, deflating or inflating the inflatable member such that the pressure achieves the target inflated pressure value.

Clause 2. The inflatable medical device of clause 1, wherein the threshold level is a first threshold level, wherein the operations comprise: setting the first threshold level and a second threshold level based on the target inflated pressure value; in response to the pressure being detected as equal to or greater than the first threshold level, deflating the inflatable member such that the pressure achieves the target inflated pressure value or is between the first threshold level and the second threshold level; and in response to the pressure being detected as less than the second threshold level for at least the period of time, inflating the inflatable member such that the pressure achieves the target inflated pressure value or is between the first threshold level and the second threshold level.

Clause 3. The inflatable medical device of clause 1 or 2, wherein the threshold level is a first threshold level, and the period of time is a first period of time, wherein the operations comprise: receiving a target deflated pressure value; determining a second threshold level for a deflate state based on the target deflated pressure value; in response to a deflate signal, deflating the inflatable member to the target deflated pressure value; detecting whether the pressure of the inflatable member achieves the second threshold level for at least a second period of time; and in response to the pressure of the inflatable member achieving the second threshold level for at least the second period of time, deflating or inflating the inflatable member such that the pressure achieves the target deflated pressure value.

Clause 4. The inflatable medical device of any one of clauses 1 to 3, wherein the operations comprising: detecting a forced deflate event; and in response to detection of the forced deflate event, deflating the inflatable member to a target deflated pressure value or a lower pressure value.

Clause 5. The inflatable medical device of any one of clauses 1 to 4, wherein the operations comprising: detecting a presence of a magnetic field caused by a device charger; and in response to the presence of the magnetic field being detected, switching an operating mode of the electronic pump device from a storage mode to an active mode.

Clause 6. The inflatable medical device of any one of clauses 1 to 5, wherein the operations comprising: initiating a countdown timer; and in response to detecting expiration of the countdown timer, switching an operating mode of the electronic pump device to an end-of-life mode, wherein the inflate state is disabled in response to the electronic pump device being configured in the end-of-life mode.

Clause 7. The inflatable medical device of any one of clauses 1 to 6, further comprising: a controller configured to wirelessly communicate with the electronic pump device, the controller including an application executable by an external device, the application configured to: detect a user command; and in response to the user command being detected, transmit the inflate signal or a deflate signal to the electronic pump device.

Clause 8. The inflatable medical device of clause 7, wherein the application is configured to: display a user interface with a device control on a display of the external device; and in response to a selection to the device control, detect the user command.

Clause 9. The inflatable medical device of any one of clauses 1 to 8, further comprising: a controller configured to wirelessly communicate with the electronic pump device, the controller including an application executable by an external device, the application configured to: display a user interface on a display of the external device; receive, via the user interface, a user-defined setting; and transmit the user-defined setting to the electronic pump device.

Clause 10. The inflatable medical device of any one of clauses 1 to 9, further comprising: a provider application executable by an external device, the provider application configured to transmit a software update to a provider platform, the provider platform configured to initiate installation of the software update on the electronic pump device.

Clause 11. A method comprising: receiving a target inflated pressure value; in response to an inflate signal to initiate an inflate state, inflating an inflatable member to the target inflated pressure value; determining a threshold level based on the target inflated pressure value; detecting, during the inflate state, whether a pressure of the inflatable member achieves the threshold level for at least a period of time; and in response to the pressure of the inflatable member achieving the threshold level for at least the period of time, deflating or inflating the inflatable member such that the pressure achieves the target inflated pressure value.

Clause 12. The method of clause 11, wherein the threshold level is a first threshold level, the method further comprising: setting the first threshold level and a second threshold level based on the target inflated pressure value; in response to the pressure being detected as equal to or greater than the first threshold level for at least the period of time, deflating the inflatable member such that the pressure achieves the target inflated pressure value or is between the first threshold level and the second threshold level; and in response to the pressure being detected as less than the second threshold level for at least the period of time, inflating the inflatable member such that the pressure achieves the target inflated pressure value or is between the first threshold level and the second threshold level.

Clause 13. The method of clause 11 or 12, wherein the threshold level is a first threshold level, and the period of time is a first period of time, the method further comprising: receiving a target deflated pressure value; determining a second threshold level for a deflate state based on the target deflated pressure value; in response to a deflate signal, deflating the inflatable member to the target deflated pressure value; detecting whether the pressure of the inflatable member achieves the second threshold level for at least a second period of time; and in response to the pressure of the inflatable member achieving the second threshold level for at least the second period of time, deflating or inflating the inflatable member such that the pressure achieves the target deflated pressure value or is between the first threshold level and the second threshold level.

Clause 14. An inflatable medical device comprising: an electronic pump device configured to transfer fluid between an inflatable member and a fluid reservoir, the electronic pump device configured to execute operations, the operations comprising: receiving a target deflated pressure value; in response to a deflate signal to initiate a deflate state, deflating the inflatable member to the target deflated pressure value; determining a threshold level based on the target deflated pressure value; detecting, during the deflate state, whether a pressure of the inflatable member achieves the threshold level for at least a period of time; and in response to the pressure of the inflatable member achieving the threshold level for at least the period of time, deflating or inflating the inflatable member such that the pressure achieves the target deflated pressure value.

Clause 15. The inflatable medical device of clause 14, wherein the threshold level is a first threshold level, wherein the operations comprise: setting the first threshold level and a second threshold level based on the target deflated pressure value; in response to the pressure being detected as equal to or greater than the first threshold level for at least the period of time, deflating the inflatable member such that the pressure achieves the target deflated pressure value or is between the first threshold level and the second threshold level; and in response to the pressure being detected as less than the second threshold level for at least the period of time, inflating the inflatable member such that the pressure achieves the target deflated pressure value or is in between the first threshold level and the second threshold level.

Clause 16. An inflatable medical device comprising: an electronic pump device configured to transfer fluid between an inflatable member and a fluid reservoir, the electronic pump device configured to execute operations, the operations comprising: receiving a target inflated pressure value; in response to an inflate signal to initiate an inflate state, inflating the inflatable member to the target inflated pressure value; determining a threshold level based on the target inflated pressure value; detecting, during the inflate state, whether a pressure of the inflatable member achieves the threshold level for at least a period of time; and in response to the pressure of the inflatable member achieving the threshold level for at least the period of time, deflating or inflating the inflatable member such that the pressure achieves the target inflated pressure value.

Clause 17. The inflatable medical device of clause 16, wherein the threshold level is a first threshold level, wherein the operations comprise: setting the first threshold level and a second threshold level based on the target inflated pressure value; in response to the pressure being detected as equal to or greater than the first threshold level, deflating the inflatable member such that the pressure achieves the target inflated pressure value or is between the first threshold level and the second threshold level; and in response to the pressure being detected as less than the second threshold level for at least the period of time, inflating the inflatable member such that the pressure achieves the target inflated pressure value or is between the first threshold level and the second threshold level.

Clause 18. The inflatable medical device of clause 16, wherein the threshold level is a first threshold level, and the period of time is a first period of time, wherein the operations comprise: receiving a target deflated pressure value; determining a second threshold level for a deflate state based on the target deflated pressure value; in response to a deflate signal, deflating the inflatable member to the target deflated pressure value; detecting whether the pressure of the inflatable member achieves the second threshold level for at least a second period of time; and in response to the pressure of the inflatable member achieving the second threshold level for at least the second period of time, deflating or inflating the inflatable member such that the pressure achieves the target deflated pressure value.

Clause 19. The inflatable medical device of clause 16, wherein the operations comprising: detecting a forced deflate event; and in response to detection of the forced deflate event, deflating the inflatable member to a target deflated pressure value or a lower pressure value.

Clause 20. The inflatable medical device of clause 19, wherein detecting the forced deflate event includes: detecting a disconnection to a wireless connection between a controller and the electronic pump device.

Clause 21. The inflatable medical device of clause 16, wherein the operations comprising: detecting a presence of a magnetic field caused by a device charger; and in response to the presence of the magnetic field being detected, switching an operating mode of the electronic pump device from a storage mode to an active mode.

Clause 22. The inflatable medical device of clause 16, wherein the operations comprising: initiating a countdown timer; and in response to detecting expiration of the countdown timer, switching an operating mode of the electronic pump device to an end-of-life mode, wherein the inflate state is disabled in response to the electronic pump device being configured in the end-of-life mode.

Clause 23. The inflatable medical device of clause 16, further comprising: a controller configured to wirelessly communicate with the electronic pump device, the controller including an application executable by an external device, the application configured to: detect a user command; and in response to the user command being detected, transmit the inflate signal or a deflate signal to the electronic pump device.

Clause 24. The inflatable medical device of clause 23, wherein the application is configured to: display a user interface with a device control on a display of the external device; and in response to a selection to the device control, detect the user command.

Clause 25. The inflatable medical device of clause 23, wherein the application is configured to: receive an audio signal; and detect the user command from the audio signal.

Clause 26. The inflatable medical device of clause 16, further comprising: a controller configured to wirelessly communicate with the electronic pump device, the controller including an application executable by an external device, the application configured to: display a user interface on a display of the external device; receive, via the user interface, a user-defined setting; and transmit the user-defined setting to the electronic pump device.

Clause 27. The inflatable medical device of clause 26, further comprising: a management agent executable by the external device, the management agent configured to: receive, over a network, enforcement policies from a provider platform; and apply the enforcement policies to manage one or more computer functions of the external device.

Clause 28. The inflatable medical device of clause 16, further comprising: a provider application executable by an external device, the provider application configured to transmit a software update to a provider platform, the provider platform configured to initiate installation of the software update on the electronic pump device.

Clause 29. The inflatable medical device of clause 28, wherein the provider application is configured to: initiate deactivation of a patient application executable by a first external device; and initiate deactivation of a clinician application executable by a second external device.

Clause 30. A method comprising: receiving a target inflated pressure value; in response to an inflate signal to initiate an inflate state, inflating an inflatable member to the target inflated pressure value; determining a threshold level based on the target inflated pressure value; detecting, during the inflate state, whether a pressure of the inflatable member achieves the threshold level for at least a period of time; and in response to the pressure of the inflatable member achieving the threshold level for at least the period of time, deflating or inflating the inflatable member such that the pressure achieves the target inflated pressure value.

Clause 31. The method of clause 30, wherein the threshold level is a first threshold level, the method further comprising: setting the first threshold level and a second threshold level based on the target inflated pressure value; in response to the pressure being detected as equal to or greater than the first threshold level for at least the period of time, deflating the inflatable member such that the pressure achieves the target inflated pressure value or is between the first threshold level and the second threshold level; and in response to the pressure being detected as less than the second threshold level for at least the period of time, inflating the inflatable member such that the pressure achieves the target inflated pressure value or is between the first threshold level and the second threshold level.

Clause 32. The method of clause 30, wherein the threshold level is a first threshold level, and the period of time is a first period of time, the method further comprising: receiving a target deflated pressure value; determining a second threshold level for a deflate state based on the target deflated pressure value; in response to a deflate signal, deflating the inflatable member to the target deflated pressure value; detecting whether the pressure of the inflatable member achieves the second threshold level for at least a second period of time; and in response to the pressure of the inflatable member achieving the second threshold level for at least the second period of time, deflating or inflating the inflatable member such that the pressure achieves the target deflated pressure value or is between the first threshold level and the second threshold level.

Clause 33. The method of clause 30, further comprising: detecting a forced deflate event; and in response to detection of the forced deflate event, deflating the inflatable member to a target deflated pressure value or a lower pressure value.

Clause 34. An inflatable medical device comprising: an electronic pump device configured to transfer fluid between an inflatable member and a fluid reservoir, the electronic pump device configured to execute operations, the operations comprising: receiving a target deflated pressure value; in response to a deflate signal to initiate a deflate state, deflating the inflatable member to the target deflated pressure value; determining a threshold level based on the target deflated pressure value; detecting, during the deflate state, whether a pressure of the inflatable member achieves the threshold level for at least a period of time; and in response to the pressure of the inflatable member achieving the threshold level for at least the period of time, deflating or inflating the inflatable member such that the pressure achieves the target deflated pressure value.

Clause 35. The inflatable medical device of clause 34, wherein the threshold level is a first threshold level, wherein the operations comprise: setting the first threshold level and a second threshold level based on the target deflated pressure value; in response to the pressure being detected as equal to or greater than the first threshold level for at least the period of time, deflating the inflatable member such that the pressure achieves the target deflated pressure value or is between the first threshold level and the second threshold level; and in response to the pressure being detected as less than the second threshold level for at least the period of time, inflating the inflatable member such that the pressure achieves the target deflated pressure value or is in between the first threshold level and the second threshold level.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An inflatable medical device comprising:
an electronic pump device configured to transfer fluid between an inflatable member and a fluid reservoir, the electronic pump device configured to execute operations, the operations comprising:
receiving a target inflated pressure value;
in response to an inflate signal to initiate an inflate state, inflating the inflatable member to the target inflated pressure value;
determining a threshold level based on the target inflated pressure value;
detecting, during the inflate state, whether a pressure of the inflatable member achieves the threshold level for at least a period of time; and
in response to the pressure of the inflatable member achieving the threshold level for at least the period of time, deflating or inflating the inflatable member such that the pressure achieves the target inflated pressure value.

2. The inflatable medical device of claim 1, wherein the threshold level is a first threshold level, wherein the operations comprise:
setting the first threshold level and a second threshold level based on the target inflated pressure value;
in response to the pressure being detected as equal to or greater than the first threshold level, deflating the inflatable member such that the pressure achieves the target inflated pressure value or is between the first threshold level and the second threshold level; and
in response to the pressure being detected as less than the second threshold level for at least the period of time, inflating the inflatable member such that the pressure achieves the target inflated pressure value or is between the first threshold level and the second threshold level.

3. The inflatable medical device of claim 1, wherein the threshold level is a first threshold level, and the period of time is a first period of time, wherein the operations comprise:
receiving a target deflated pressure value;
determining a second threshold level for a deflate state based on the target deflated pressure value;
in response to a deflate signal, deflating the inflatable member to the target deflated pressure value;
detecting whether the pressure of the inflatable member achieves the second threshold level for at least a second period of time; and
in response to the pressure of the inflatable member achieving the second threshold level for at least the second period of time, deflating or inflating the inflatable member such that the pressure achieves the target deflated pressure value.

4. The inflatable medical device of claim 1, wherein the operations comprising:
detecting a forced deflate event; and
in response to detection of the forced deflate event, deflating the inflatable member to a target deflated pressure value or a lower pressure value.

5. The inflatable medical device of claim 4, wherein detecting the forced deflate event includes:
detecting a disconnection to a wireless connection between a controller and the electronic pump device.

6. The inflatable medical device of claim 1, wherein the operations comprising:
detecting a presence of a magnetic field caused by a device charger; and
in response to the presence of the magnetic field being detected, switching an operating mode of the electronic pump device from a storage mode to an active mode.

7. The inflatable medical device of claim 1, wherein the operations comprising:
initiating a countdown timer; and
in response to detecting expiration of the countdown timer, switching an operating mode of the electronic pump device to an end-of-life mode, wherein the inflate state is disabled in response to the electronic pump device being configured in the end-of-life mode.

8. The inflatable medical device of claim 1, further comprising:
a controller configured to wirelessly communicate with the electronic pump device, the controller including an application executable by an external device, the application configured to:
detect a user command; and
in response to the user command being detected, transmit the inflate signal or a deflate signal to the electronic pump device.

9. The inflatable medical device of claim 8, wherein the application is configured to:
display a user interface with a device control on a display of the external device; and
in response to a selection to the device control, detect the user command.

10. The inflatable medical device of claim 8, wherein the application is configured to:
receive an audio signal; and
detect the user command from the audio signal.

11. The inflatable medical device of claim 1, further comprising:
a controller configured to wirelessly communicate with the electronic pump device, the controller including an application executable by an external device, the application configured to:
display a user interface on a display of the external device;
receive, via the user interface, a user-defined setting; and
transmit the user-defined setting to the electronic pump device.

12. The inflatable medical device of claim 11, further comprising:
a management agent executable by the external device, the management agent configured to:
receive, over a network, enforcement policies from a provider platform; and
apply the enforcement policies to manage one or more computer functions of the external device.

13. The inflatable medical device of claim 1, further comprising:
a provider application executable by an external device, the provider application configured to transmit a software update to a provider platform, the provider platform configured to initiate installation of the software update on the electronic pump device.

14. The inflatable medical device of claim 13, wherein the provider application is configured to:
initiate deactivation of a patient application executable by a first external device; and
initiate deactivation of a clinician application executable by a second external device.

15. A method comprising:
receiving a target inflated pressure value;
in response to an inflate signal to initiate an inflate state, inflating an inflatable member to the target inflated pressure value;
determining a threshold level based on the target inflated pressure value;
detecting, during the inflate state, whether a pressure of the inflatable member achieves the threshold level for at least a period of time; and
in response to the pressure of the inflatable member achieving the threshold level for at least the period of time, deflating or inflating the inflatable member such that the pressure achieves the target inflated pressure value.

16. The method of claim 15, wherein the threshold level is a first threshold level, the method further comprising:
setting the first threshold level and a second threshold level based on the target inflated pressure value;
in response to the pressure being detected as equal to or greater than the first threshold level for at least the period of time, deflating the inflatable member such that the pressure achieves the target inflated pressure value or is between the first threshold level and the second threshold level; and
in response to the pressure being detected as less than the second threshold level for at least the period of time, inflating the inflatable member such that the pressure achieves the target inflated pressure value or is between the first threshold level and the second threshold level.

17. The method of claim 15, wherein the threshold level is a first threshold level, and the period of time is a first period of time, the method further comprising:
receiving a target deflated pressure value;
determining a second threshold level for a deflate state based on the target deflated pressure value;
in response to a deflate signal, deflating the inflatable member to the target deflated pressure value;
detecting whether the pressure of the inflatable member achieves the second threshold level for at least a second period of time; and
in response to the pressure of the inflatable member achieving the second threshold level for at least the second period of time, deflating or inflating the inflatable member such that the pressure achieves the target deflated pressure value or is between the first threshold level and the second threshold level.

18. The method of claim 15, further comprising:
detecting a forced deflate event; and
in response to detection of the forced deflate event, deflating the inflatable member to a target deflated pressure value or a lower pressure value.

19. An inflatable medical device comprising:
an electronic pump device configured to transfer fluid between an inflatable member and a fluid reservoir, the electronic pump device configured to execute operations, the operations comprising:
receiving a target deflated pressure value;
in response to a deflate signal to initiate a deflate state, deflating the inflatable member to the target deflated pressure value;
determining a threshold level based on the target deflated pressure value;
detecting, during the deflate state, whether a pressure of the inflatable member achieves the threshold level for at least a period of time; and
in response to the pressure of the inflatable member achieving the threshold level for at least the period of time, deflating or inflating the inflatable member such that the pressure achieves the target deflated pressure value.

20. The inflatable medical device of claim 19, wherein the threshold level is a first threshold level, wherein the operations comprise:
setting the first threshold level and a second threshold level based on the target deflated pressure value;
in response to the pressure being detected as equal to or greater than the first threshold level for at least the period of time, deflating the inflatable member such that the pressure achieves the target deflated pressure value or is between the first threshold level and the second threshold level; and
in response to the pressure being detected as less than the second threshold level for at least the period of time, inflating the inflatable member such that the pressure achieves the target deflated pressure value or is in between the first threshold level and the second threshold level.
